(19) United States
(12) Patent Application Publication
(10) Pub. No.: US 2010/0161002 A1
(43) Pub. Date: Jun. 24, 2010

(54) Implantable Medical Device Having A Slot Antenna In Its Case

(75) Inventors: Daniel Aghassian, Los Angeles, CA (US); Lev Freidin, Simi Valley, CA (US); Vasily Dronov, Valencia, CA (US)

Correspondence Address: Wong, Cabello, Lutsch, Rutherfor & Brucculer L.L.P, 20333 SH 249, Suite 600, Houston, TX, 77070, US

(73) Assignee: Boston Scientific Neuromodulation Corporation, Valencia, CA (US)

(21) Appl. No.: 12/341,726

(22) Filed: Dec. 22, 2008

(51) Int. Cl. A61N 1/372

(52) U.S. Cl. 607/60

(57) ABSTRACT

Disclosed is an improved medical implantable device having a conductive case into which a slot antenna is formed. The slot antenna preferably has a slot length which is one-half of the wavelength of the data being sent to or received from an external controller, although slot lengths smaller than these ideals values can also be used albeit with reduced efficiency. Slot lengths accommodatable by a given case can enable communications at frequencies suitable for medical telemetry. The slot is preferably filled with a hermetic dielectric material, and can be formed into different geometries, including non-linear geometries. When the slot antenna is provided in the implant's case, separate data antennas or coils are not needed, which reduces the implant's size. Additionally, the slot antenna reduces eddy current heating in the case, and promotes efficient data transfer in the near field that is not as susceptible to attenuation in the human body.

### FIELD OF THE INVENTION

The present invention relates to an improved implantable medical device having a slot antenna formed in its conductive casing.

### BACKGROUND

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder sublaxation, etc. The description that follows will generally focus on the use of the invention within a Spinal Cord Stimulation (SCS) system, such as that disclosed in U.S. Pat. No. 6,516,227. However, the present invention may find applicability in any implantable medical device system. For example, the disclosed invention can also be used with a Bion™ implantable stimulator, such as is shown in U.S. Patent Publication 2007/0097719, filed Nov. 3, 2005, or with other implantable medical devices.

As shown in FIGS. 1A and 1B, a SCS system typically includes an Implantable Pulse Generator (IPG) 100, which includes a biocompatible device case 30 formed of titanium for example. The case 30 typically holds the circuitry and battery 26 necessary for the IPG to function, although IPGs can also be powered via external RF energy and without a battery. The IPG 100 is coupled to electrodes 106 via one or more electrode leads (two such leads 102 and 104 are shown), such that the electrodes 106 form an electrode array 110. The electrodes 106 are carried on a flexible body 108, which also houses the individual signal wires 112 and 114 coupled to each electrode. In the illustrated embodiment, there are eight electrodes on lead 102, labeled E₁-E₈, and eight electrodes on lead 104, labeled E₉-E₁₆, although the number of leads and electrodes is application specific and therefore can vary. The leads 102, 104 couple to the IPG 100 using lead connectors 38a and 38b, which are fixed in a header material 36, which can comprise an epoxy for example.

As shown in FIG. 2, the IPG 100 typically includes an electronic substrate assembly 14 including a printed circuit board (PCB) 16, along with various electronic components 20, such as microprocessors, integrated circuits, and capacitors mounted to the PCB 16. Two coils are generally present in the IPG 100: a telemetry coil 13 used to transmit/receive data to/from an external controller 12; and a charging coil 18 for charging or recharging the IPG's battery 26 using an external charger 50. The telemetry coil 13 can be mounted within the header 36 of the IPG 100 as shown.

As just noted, an external controller 12, such as a hand-held programmer or a clinician's programmer, is used to send data to and receive data from the IPG 100. For example, the external controller 12 can send programming data to the IPG 100 to dictate the therapy the IPG 100 will provide to the patient. Also, the external controller 12 can act as a receiver of data from the IPG 100, such as various data reporting on the IPG's status. The external controller 12, like the IPG 100, also contains a PCB 70 on which electronic components 72 are placed to control operation of the external controller 12. A user interface 74 similar to that used for a computer, cell phone, or other hand held electronic device, and including touchable buttons and a display for example, allows a patient or clinician to operate the external controller 12.

Wireless data transfer between the IPG 100 and the external controller 12 takes place via inductive coupling. To implement such functionality, both the IPG 100 and the external controller 12 have coils 13 and 17 respectively. Either coil can act as the transmitter or the receiver, thus allowing for two-way communication between the two devices. When data is to be sent from the external controller 12 to the IPG 100, coil 17 is energized with alternating current (AC), which generates a magnetic field 29, which in turn induces a voltage in the IPG's telemetry coil 13. The generated magnetic field 29 is typically modulated using a communication protocol, such as a Frequency Shift Keying (FSK) protocol, which is well known in the art. The power used to energize the coil 17 can come from a battery 76, which like the IPG's battery 26 is preferably rechargeable, but power may also come from plugging the external controller 12 into a wall outlet plug (not shown), etc. The induced voltage in coil 13 can then be demodulated at the IPG 100 back into the telemetered data signals. To improve the magnetic flux density, and hence the efficiency of the data transfer, the IPG's telemetry coil 13 may be wrapped around a ferrite core 13′.

The external charger 50 is used to charge (or recharge) the IPG's battery 26. Specifically, and similarly to the external controller, the coil 17′ is energized with an AC current to create a magnetic field 29. This magnetic field 29 induces a current in the charging coil 18 within the IPG 100, which current is rectified to DC levels, and used to recharge the battery 26. The external charger 50 will generally have many of the same basic components as the external controller 12, and therefore have similar element numerals, denoted with prime symbols. However, while sufficient for purposes of this disclosure to view the external controller 12 and charger 50 as essentially the same, one skilled in the art will realize that external controllers 12 and chargers 50 will have pertinent differences as dictated by their respective functions.

As is well known, inductive transmission of data or power can occur transcutaneously, i.e., through the patient's tissue 25, making it particular useful in a medical implantable device system. During the transmission of data or power, the coils 13 and 17, or 18 and 17′, preferably lie along a common axis in planes that are parallel. Such an orientation between the coils will generally improve the coupling between them, but deviation from ideal orientations can still result in suitably reliable data or power transfer.

It is desirable to make the IPG 100 as small as possible to reduce the inconvenience to the patient in which the IPG is implanted. Additionally, the IPG 100 should be simple to manufacture and reliable in its operation. In this regard, the inventors find the need for a communication coil 13 unfortunate. The communication coil 13 takes up room in the header 36, which increases the overall size of the IPG 100. Additionally, the communication coil 13 requires special care during manufacture. First, the coil 13′ must be wrapped around the ferrite core 13′. Then it must be connected to the electronic substrate assembly 14. This requires the provisional of a hermetic feedthrough in the IPG case 30. Then the communication coil assembly must be encapsulated in the header 36 material. All of these manufacturing steps are relatively complex and can give rise to reliability concerns.

Given these shortcomings, the art of implantable medical devices would benefit from an improved communication transmission/reception device for an implantable medical device, and this disclosure presents such a solution.

### BRIEF DESCRIPTION OF THE DRAWINGS

FIGS. 1A and 1B show an implantable pulse generator (IPG), and the manner in which an electrode array is coupled to the IPG in accordance with the prior art.

FIG. 2 shows the relation between the IPG and an external controller and an external charger.

FIGS. 3A and 3B show an improved IPG having a slot antenna formed in its body.

FIGS. 4A and 4B show transmission and reception circuitry useable in conjunction with the slot antenna of FIGS. 3A and 3B.

FIGS. 5A-5C show various geometries for the slot antenna(s) in the improved IPG.

### DETAILED DESCRIPTION

The description that follows relates to use of the invention within a spinal cord stimulation (SCS) system. However, it is to be understood that the invention is not so limited. Rather, the invention may be used with any type of implantable medical device that could benefit from an improved communication antenna. For example, the present invention may be used as part of a system employing an implantable sensor, an implantable pump, a pacemaker, a defibrillator, a cochlear stimulator, a retinal stimulator, a stimulator configured to produce coordinated limb movement, a cortical and deep brain stimulator, or in any other neural stimulator configured to treat any of a variety of conditions.

Disclosed is an improved medical implantable device having a conductive case into which a slot antenna is formed. The slot antenna preferably has a slot length which is one-half of the wavelength of the data being sent to or received from an external controller, although slot lengths smaller than these ideals values can also be used albeit with reduced efficiency. Slot lengths accommodatable by a given case can enable communications at frequencies suitable for medical telemetry. The slot is preferably filled with a hermetic dielectric material, and can be formed into different geometries, including non-linear geometries. When the slot antenna is provided in the implant's case, separate data antennas or coils are not needed, which reduces the implant's size. Additionally, the slot antenna reduces eddy current heating in the case, and promotes efficient data transfer in the near field that is not as susceptible to attenuation in the human body.

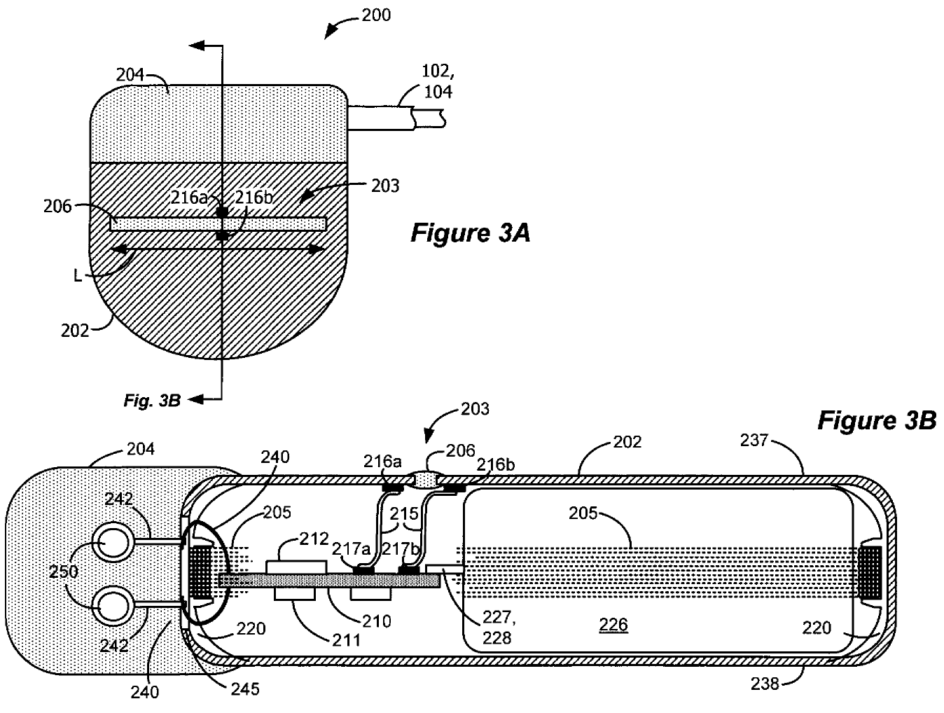

FIGS. 3A and 3B show an improved IPG 200 having a slot antenna 203 in plan and cross-sectional views. However, before discussing the slot antenna 203 and its benefits, other structures present in the improved IPG 200 are discussed for completeness.

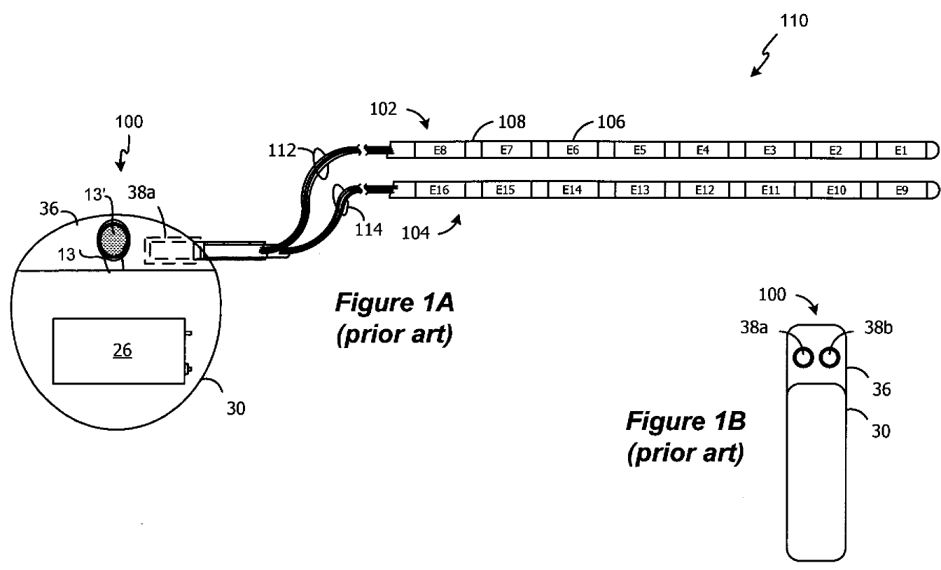

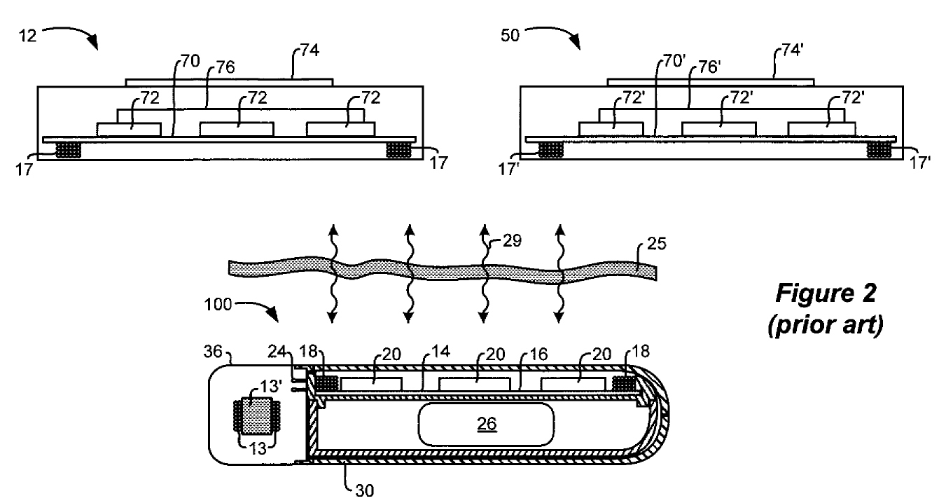

The IPG 200 includes a dielectric header 204 formed of medical-grade epoxy for example, which houses lead connectors 250 for meeting with leads 102, 104 of an electrode array 110 (see FIG. 1A). The IPG 200 also includes a metallic case 202 which houses, among other structures, a coil 205, the rechargeable battery 226, and a printed circuit board (PCB) 210 which is coupled to both. As one skilled in the art will understand, the PCB 210 will support the electronics 212 (e.g., the microcontroller, memory, rectifiers, regulators, current sources, etc.) necessary for the IPG 200 to operate. The coil 205, PCB 210, and battery 226 can be positioned in the case 202 using an internal support 220, which may be made of plastic for example. In the embodiment shown, the PCB 210 is double sided, with the underside of the PCB supporting the output capacitors 211 associated with each of the stimulation electrodes 106 (FIG. 1A) of the IPG 200. The terminals 227 and 228 of the battery 226 are shown as soldered to the top side of the PCB 210. Flexible circuits 240 are used to connect the PCB 210 to the lead connectors 250, and ultimately to the electrodes 106 in the electrode array 110 (FIG. 1A). The flexible circuits are soldered at one end to contacts of the PCB 210, and are soldered to pins 242, each of which fit into an appropriate lead connector 250 for an electrode. The flexible circuit 240 can be bent around the coil 205 and passed though a feedthrough 245 in the case 202 and into the header 204. In this embodiment, the coil 205 serves as the power reception coil for receiving power from the external charger 50 (FIG. 2).

The slot antenna 203 comprises a slot in the conductive case 202 of the IPG 200, or more generally a hole in the conductive case which hole need not necessarily be slot-shaped. As explained below, the slot antenna can receive data from, and transmit data to, an external controller 12. This is a beneficial addition to an implantable medical device: by providing a slot or hole in the already-present conductive case 202, data can be received and transferred without the need for an additional communication coil 13 (FIGS. 1A and 2). As a result, the manufacturing and reliability difficulties associated with the communication coil 13, discussed in the Background, are dispensed with, and the IPG 200 can be made smaller.

As illustrated, the slot antenna 203 is formed in the top side 237 of the conductive case 202. The top side 237 of the case 202 can initially be formed without a slot antenna, and then the slot antenna 203 can later be milled, cut, punched, scribed, etc. into the top side before it is brazed to the bottom side 238 during IPG manufacture. Because the slot antenna 203 must be hermetic given the IPG 200's expected environment in the human body, a hermitic dielectric material 206 is used to fill the slot antenna 203. Such material 206 may comprise glass, ceramic, or other non-conductive hermetical material, which may be brazed, hardened, or otherwise set into place in the IPG's case 202. When implanted, the slot antenna 203 is preferably placed so as to face outside the patient, which improves data transmission/reception.

The slot antenna 203 is preferably a one-half wavelength (i.e., ½λ) antenna, meaning that the length, L, of the slot antenna 203 is approximately one-half of the wavelength of the data signal to be received/transmitted at/from the antenna 203. The benefits of using a ½λ antenna are well known generally in the communication arts, and are therefore not expounded upon here.

In one embodiment, the length, L, of the slot antenna 203 is approximately one inch, but can vary. A one-inch slot length is generally easily accommodated by a typical IPG 200, whose conductive case 202 will generally have (or can be made to have) at least one dimension of at least one inch. For example, in an SCS system, the IPG 200 is generally disc shaped, and the case 202 is normally greater than one inch in diameter. Therefore, the slot antenna 203 is generally easily accommodated without further need to modify the geometry of the case 202.

Although the slot length L of the slot antenna 203 is ideally ½λ to match the wavelength of the radiation λ that it receives or transmits, this is not required. The slot length L can be bigger or smaller than such ideal values, although with lower efficiency. However, such reduced efficiency can be mitigated using a slower data throughput, a topic discussed further below.

A slot antenna 203 having a length of approximately ½λ=6 centimeters, will ideally receive/transmit radiation at a frequency of 2.4 GHz (where f=c/λ). (In reality, c equals the speed of radiation in a given media, which media for a medical implant could include both air and the human body for example. However, for simplicity, such media-induced variations in c are ignored in the calculations, and instead a vacuum value for c, i.e., 3×10⁸ m/s, is assumed). However, because such lengths may be longer than the longest dimension of the IPG's case 202, the slot length L can be made smaller than these ideal values. Such smaller slot length antennas 203 can still operate at 2.4 GHz, although with reduced performance and at lower data rates as just mentioned. A frequency of this magnitude matches the frequency specified by the Zigbee wireless standard, which is well known, and which can be used with the disclosed slot antenna 203. (Further details concerning Zigbee can be found at http://en.wikipedia.org/wiki/ZigBee, a copy of which is included with the Information Disclosure Statement filed herewith, and which is incorporated herein by reference in its entirety). The well-known Bluetooth protocol, or other protocols which operate at 2.4 GHz, could be used as well.

Communication to and from the slot antenna 203 can also occur using Frequency Shift Keying (FSK). FSK comprises a serial data stream of logic ‘0’s and ‘1’s comprising different frequencies generally centered around the target of 2.4 GHz. Thus, a logic ‘0’ might comprise a transmission having a frequency of 2.4 GHz−Δf, while a logic ‘1’ might comprise a transmission having a frequency of 2.4 GHz+Δf, where Δf is small compared to 2.4 GHz. FSK communications are discussed further in U.S. patent application Ser. No. 11/853,624, filed Sep. 11, 2007, which is incorporated herein by reference in its entirety. Other types of modulation could be used as well, including phase shift keying (PSK), Quadrature Phase shift keying (QPSK), offset QPSK (OQPSK), On-Off Keying (OOK), or other modulations schemes suitable for the frequencies being used.

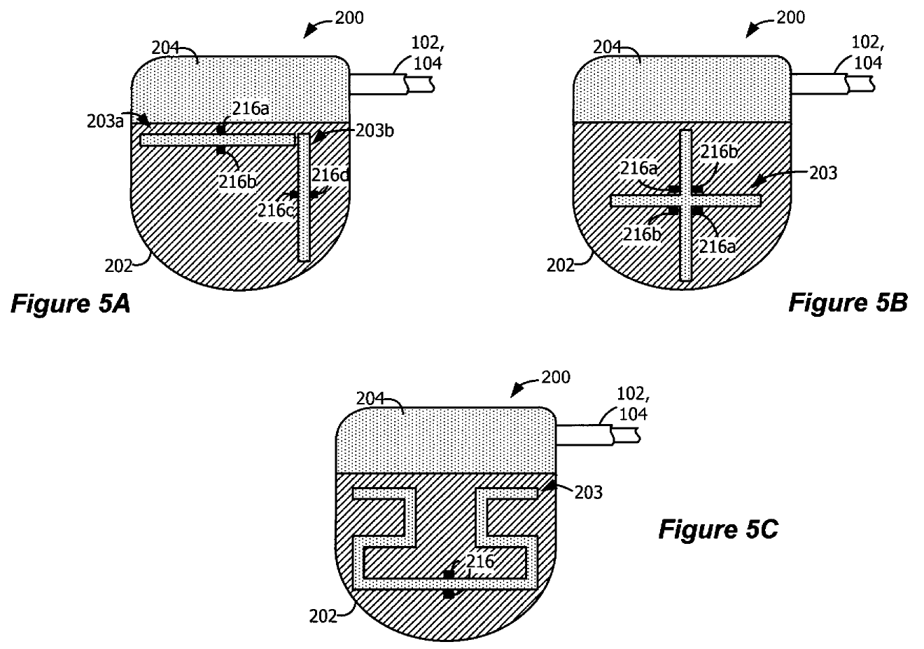

Lengthening of the slot antenna 203 will allow for the use of other communication standards that operate at even lower frequencies, such as the Medical Implant Communications Service, or MICS, which uses frequencies of approximately 405 MHz. Additionally, Industrial Scientific and Medical (ISM) band frequencies can be used as well, which have center frequencies which range from 6.78 MHz to 245 GHz. See http://en.wikipedia.org/wiki/ISM_band, which is submitted with the Information Disclosure Statement filed herewith. Of the various ISM bands, those having center frequencies of 433.92 MHz (Region 1), 915 MHz (Region 2), 2.45 GHz, and 5.8 GHz, would render ½λ slot lengths L for the slot antenna 203 which are reasonable given the typical size of the IPG case 202. If ideal slots lengths are too long to be accommodated by the cases of some medical implantable devices, the slot length can be made smaller than this ideal value and operate at a reduced efficiency and at lower data rates. Moreover, a slot of a non-linear geometry can be used to improve the effective slot length. See, e.g., FIG. 5C. Such techniques are generally known in the communication arts.

As mentioned before, the length, L, of the slot antenna 203 need not exactly correspond to ½λ of the radiation used to communicate between the external controller 12 and the IPG 200. Even if the slot length L does not exactly match the communicative radiation, it can still be sufficient to receive/transmit data at/from the IPG 200, although such reception/transmission may occur at a lower efficiency. Lower efficiency may require an increase in the power of the transmitter, but such increase in power is generally acceptable, particularly when one considers that the power involved in data transmission is relatively low. Moreover, suitable communication protocols such as those mentioned earlier generally occur at data rates which are relatively low. A low data rate is generally acceptable in an implantable medical device system, which typically needs to communicate only a finite number of parameters on a periodic and non-time-critical basis. A lower data rate generally allows the spectral density of the transmitted signal to be higher, and therefore improves the system's signal-to-noise ratio. This increases the system's communication range, and allows the system to better tolerate a smaller-than-ideal slot length, L.

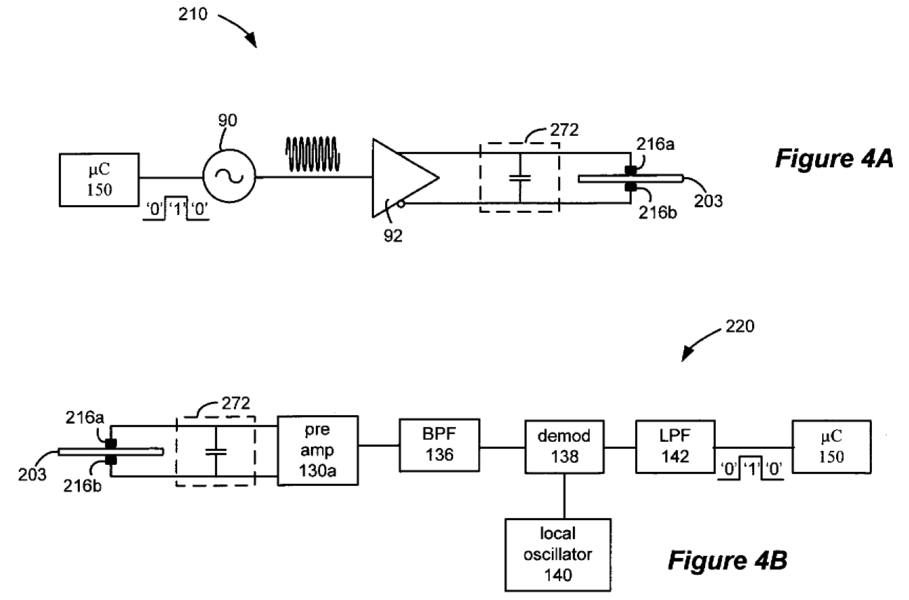

Connections to the slot antenna 203 can generally be made as shown in FIGS. 3A and 3B. Shown are connections between the slot antenna 203 and the printed circuit board 210 which supports the IPG 200's main electrical components, including the transmission and reception circuitry, which is further discussed below with reference to FIGS. 4A and 4B. Connection is preferably made using a flexible circuit 215, which may comprise a flexible Kapton-based substrate for example. Alternatively, flexible circuit 215 may comprise a nickel strip. Such flexible circuits 215 can interface with case 202 at contacts 216a and 216b, and with the PCB 210 at contacts 217a and 217b. The contacts 216a/b and 217a/b may comprise Kovar™ for example, and may be laser welded into place at the middle of the slot.

Transmission 210 and reception 220 circuitry for sending/receiving data from/to the slot antenna 203 is shown in FIGS. 4A and 4B respectively. Transmission circuitry 210 comprises a modulator 90, which may modulate the data using FSK for example. The modulated data is sent to a differential amplifier 92, whose outputs couple to the slot antenna 203. As is known in the art, an impedance matching network 272 can also be coupled to the slot to promote efficient transfer of the differential signal to the slot 203. Use of an impedance matching network can be especially important if the slot length L varies from the ideal ½λ value for the frequency being used. The impedance network 272 will vary depending on the other impedances present in the circuit, and for simplicity is merely shown as a single capacitor in the Figures. The reception circuitry 220 likewise can include an impedance matching network 272, and contains other standard circuits for demodulating the received signals. Because much of the circuitry in FIGS. 4A and 4B is discussed in the above-incorporated '624 application, it is not further discussed here.

FIGS. 5A-5C show different geometries for the slot antenna 203. FIG. 5A shows two individual slot antennas 203a and 203b having orthogonal portions and their respective case contacts 216a/b and 216c/d. Because the slots are orthogonal, they are more apt to pick up transmissions from the external controller 12, which can be particularly important if the external controller 12 is not well aligned with the IPG 200. In this embodiment, the transmission/reception circuitry within the IPG 200 modulates/demodulates the data out of phase, e.g., with a 90-degree phase difference at each of the slots 203a and 203b. Transmission/reception circuitry useable to provide such a 90-degree phase difference can be found in the above-incorporated '624 application.

FIG. 5B shows a single slot antenna 203 with intersecting portions shaped as a cross. Like the slot antennas 203a and 203b of FIG. 5A, the orthogonal nature of the cross-shaped slot antenna 203 of FIG. 5B improves coupling between the external controller 12 and the IPG 200. Transmission/reception circuitry like that depicted in FIGS. 4A and 4B can be used. Each of the contacts 216a and 216b is preferably replicated at diagonals as shown, to provide a reference on both sides of the cross. However, the two contacts 216a would be shorted together, and likewise for the contacts 216b.

FIG. 5C shows that the slot antenna 203 can take on shapes that are non-linear. By taking on non-linear shapes, the effective length of the slot 203 can be increased. Such an increased slot length assists the slot antenna 203 to transmit and receive data at lower frequencies, which increases the number of communication protocols useable with the improved IPG 200.

The slot antenna(s) 203 provides other benefits not yet mentioned. For instance, because the slot(s) interrupts the conductive plane otherwise provided by the case 202, eddy currents in the case are reduced. Reduction of eddy currents is particularly beneficial in reducing implant heating while charging the implant using the external charger 50. This, among other benefits, improves the implant's safety.

Additionally, because a slot antenna is mostly magnetic in the near field, i.e., less than approximately 10 centimeters or more generally one wavelength, data transmission is rendered more efficient. This is because magnetic fields are not as heavily attenuated in the human body as are the electromagnetic fields prevalent in the far field. As a result, transmission power can be reduced. Such attenuation reduction can additionally help to assist in overcoming any previously-noted mismatches between the slot length L and the frequency of the data signal, in so far as reduced attenuation saves transmission power useable to overcome such mismatch.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An implantable medical device, comprising:
a conductive case for housing electronic circuitry necessary for the operation of the implantable medical device; and
a slot antenna formed in the conductive case, wherein the slot antenna receives data from, transmits data to, or receives and transmits data from and to, an external device.

2. The device of claim 1, wherein the slot antenna is filled with a hermetic dielectric material.

3. The device of claim 1, wherein modulated data is transmitted or received as radiation with a wavelength λ, and wherein the slot antenna has a length of approximately ½λ.

4. The device of claim 1, wherein modulated data is transmitted or received as radiation with a frequency of between approximately 405 MHz and 5.8 GHz.

5. The device of claim 1, wherein modulated data is transmitted or received as radiation in accordance with a communications protocol.

6. The device of claim 1, wherein the slot antenna has a non linear geometry.

7. The device of claim 1, wherein the slot antenna comprises intersecting portions.

8. An implantable medical device, comprising:
a conductive case for housing electronic circuitry necessary for the operation of the implantable medical device; and
an antenna comprising a hole in the conductive case, wherein the antenna receives data from, transmits data to, or receives and transmits data from and to, an external device.

9. The device of claim 8, wherein the antenna is filled with a hermetic dielectric material.

10. The device of claim 8, wherein modulated data is transmitted or received as radiation with a wavelength λ, and wherein the antenna has a length of approximately ½λ.

11. The device of claim 8, wherein modulated data is transmitted or received as radiation with a frequency of between approximately 405 MHz and 5.8 GHz.

12. An implantable medical device, comprising:
a conductive case for housing electronic circuitry necessary for the operation of the implantable medical device;
a first slot antenna formed in the conductive case; and
a second slot antenna formed in the conductive case,
wherein the first and second slot antennas contain orthogonal portions, and operate out of phase to receive data from, transmit data to, or receive and transmit data from and to, an external device.

13. The device of claim 12, wherein the first and second slot antennas have non linear geometries.

14. The device of claim 12, wherein the slot antenna is filled with a hermetic dielectric material.

15. The device of claim 12, wherein modulated data is transmitted or received as radiation with a wavelength λ, and wherein the slot antenna has a length of approximately ½λ.

16. An implantable medical device, comprising:
a conductive case for housing electronic circuitry necessary for the operation of the implantable medical device, the conductive case being generally disk shaped and having a top surface and a bottom surface; and
an antenna comprising a hole in the top surface of the conductive case, wherein the antenna is coupled to the electronic circuitry to receives data from, transmits data to, or receives and transmits data from and to, an external device.

17. The device of claim 16, wherein the antenna comprises a slot antenna.

18. The device of claim 16, wherein the slot antenna has a first side and a second side, and wherein the first and second sides are coupled to the electronic circuitry.

19. The device of claim 18, wherein the first and second sides are coupled to the electronic circuitry at the middle of the first and second sides.

20. The device of claim 16, wherein the slot antenna is hermetically sealed.

21. An implantable medical device, comprising:
a conductive case for housing electronic circuitry necessary for the operation of the implantable medical device;
a header coupled to the conductive case, wherein the header includes at least one connector for meeting with an electrode lead for stimulating tissue of a patient, the at least one connector being coupled to the electronic circuitry through a feedthrough in the conductive case; and
a slot antenna formed in the conductive case for receiving data from, transmitting data to, or receiving and transmitting data from and to, an external device.

22. The device of claim 21, wherein the header comprises a dielectric material.

23. The device of claim 21, wherein the slot antenna is filled with a hermetic dielectric material.
